(19) United States
(12) Plant Patent Application Publication
(10) Pub. No.: US 2009/0113578 P1
(43) Pub. Date: Apr. 30, 2009

(54) Grapevine Plant Named 'Cabernet Dore'

(50) ;

(76) Inventor: Lucian W. Dressel, Carrollton, IL (US)

Correspondence Address: Lucian W. Dressel;Suite 207, RR 2, Carrollton, IL, 62016, US

(21) Appl. No.: 11/978,502

(22) Filed: Oct. 30, 2007

(51) Int. Cl. A01H 5/00

(52) U.S. Cl. PLT/205

(57) ABSTRACT

A new and distinct, self rooted, variety of grapevine, “Cabernet Doré”, from a cross of “Cabernet Sauvignon” and “Norton”, which can be distinguished by its outstanding wine combined with high productivity, disease resistance, and cold hardiness superior to its acclaimed parent “Cabernet Sauvignon”.

### BOTANICAL CLASSIFICATION

Vitis spp hybrid. (“Cabernet Sauvignon” crossed with “Norton”)

### VARIETAL DENOMINATION

“Cabernet Doré”

### BACKGROUND OF THE INVENTION

Most grape varieties used for production of high quality wines around the world are of the species Vitis Vinifera. These V. Vinifera varieties, when cultivated in northern regions of the United States with a continental climate are often subject to serious injury or death from low temperatures during winter. V. Vinifera must also be grafted onto an American rootstock in order to be grown successfully. Although several wild Vitis species occur in colder regions of North America and eastern Asia, the wine made from these species generally has serious defects. Thus, a great need existed for grape plants that would combine the superior wine quality of V. Vinifera with the cold weather resistance and disease resistance of wild species yet be free of their unpleasant wild flavors. A grape breeding program conducted by Lucian W. Dressel at Davis, Calif. and at Winters, Calif. from 2000 to 2002 developed such varieties by combining various V. Vinifera with the native grape plant known as “Norton” (aka Cynthiana, aka Virginia Seedling).

### BRIEF SUMMARY OF THE INVENTION

The invention is a new and distinct variety of grape plant designated ‘Cabernet Doré’ which produced yellow to golden fruit highly suitable for white wine production, and has a combination of high wine quality, excellent cold hardiness, disease resistance, good productivity, and does not need to be grafted. It has proven to be well adapted to various states including California, Missouri, Illinois, and Kentucky. Cabernet Doré resulted from a cross of “Cabernet Sauvignon” and “Norton” made in 2001 in Winters, Calif.

Although both parents produce black skinned grapes, it is not at all unusual for crosses to be white (or visa versa) since almost all wine grapes are highly inbred and contain recessive genes from many different forbearers. One of Cabernet Sauvignon's parents is Sauvignon Blanc, a white grape, and Norton very likely has a white parent as well.

Cabernet Doré propagates moderately well from hardwood cuttings. Once rooted however the young plants quickly become established, and all Cabernet Doré plants propagated in this manner have been genetically stable, producing only white fruit with light golden juice. The vines of Cabernet Doré have an abundance of tendrils and easily adapt themselves to a high wire cordon trellis system. Canes have a drooping growth attitude and are easily combed and trained. The bud break and bloom of Cabernet Doré are very late, typically after that of both Cabernet Sauvignon and Norton. Its flowers are perfect and self fertile. Cabernet Doré vines typically set a moderate crop. The fruit of Cabernet Doré is borne on small to medium sized clusters that are tight, compact and conical to triangular in shape. The peduncles are Norton-like, being quite long. The berries are small to medium in size with a waxy bloom at maturity. Berry splitting and bunch rot have not been observed to date, nor has crown gall. In commercial vineyards on a normal spray schedule no disease problems have been noted from Black Rot, Downey Mildew, Powdery Mildew, or any other fungus disease or insects. Resistance to Pierce's Disease is unknown, but is being tested in Louisiana.

The fruit of Cabernet Doré can be fermented to produce a dry white wine that can be sold soon after finishing or barrel aged to produce a more complex wine. The wine has none of the flavors associated with wines made from either French Hybrid grapes or V. Labrusca varieties. Cabernet Doré's wine is a light pleasant golden color. The flavors are tropical, concentrated, persistent, immediate, with dried pineapple and papayas also with hints of allspice, nutmeg and an occasional whiff of banana.

Cabernet Doré is much more cold-hardy than its parent Cabernet Sauvignon and has the advantage of being self rooted so that even if the plant is killed to the ground it can be renewed from an underground sucker, unlike Cabernet Sauvignon. Unlike its other parent, Norton, its growth habits are quite orderly and manageable, and Cabernet Doré does not have to be grown on a double curtain system to be profitable.

### BRIEF DESCRIPTION OF THE FIGURES

FIG. 1 —CabDoréLeafFront—A photograph showing the front view of a Cabernet Doré leaf.

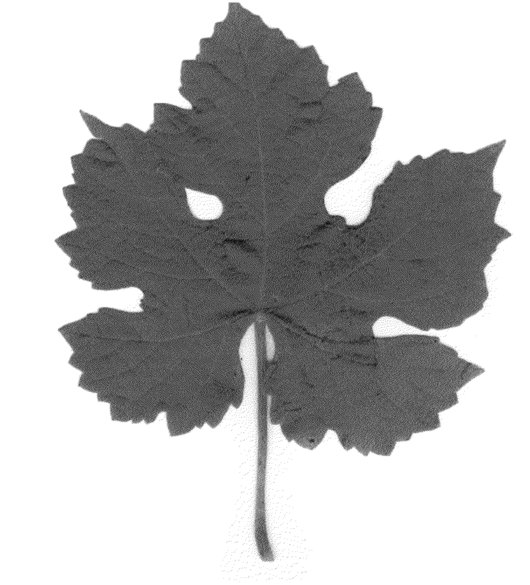

FIG. 2 —CabDoréLeafRear—A photograph showing the rear view of a Cabernet Doré leaf.

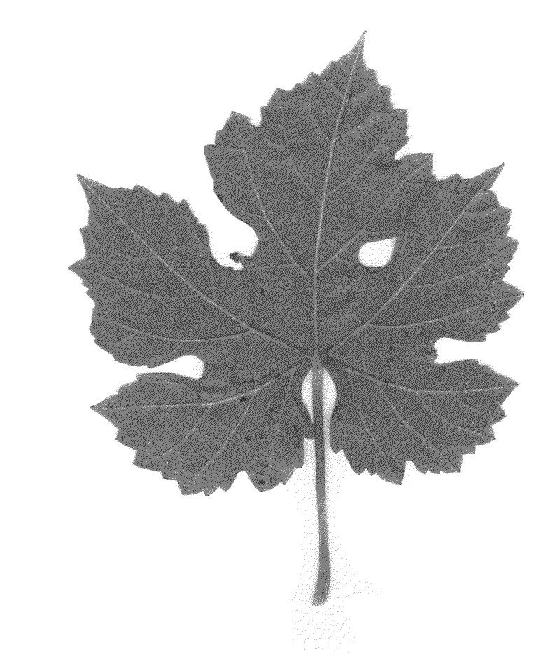

FIG. 3 —CabDoréVine—A photograph showing trunk, canes, leaves and fruit after version in 2006.

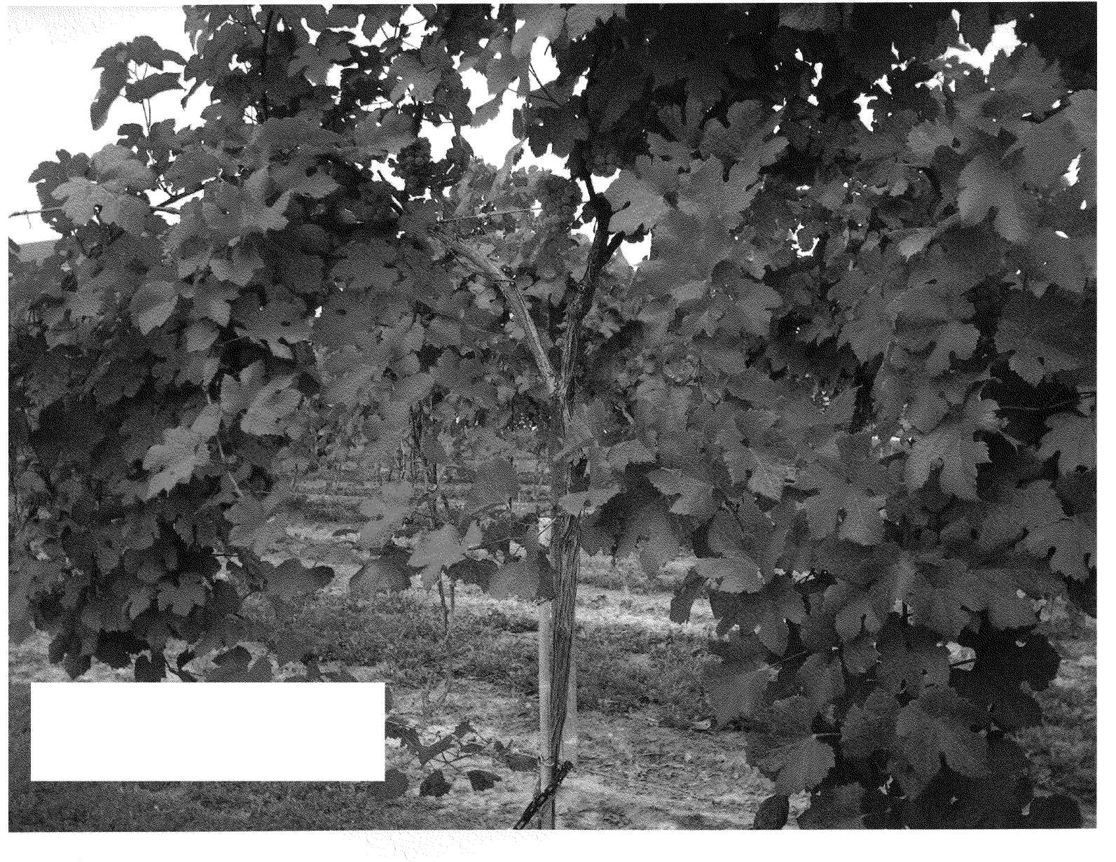

FIG. 4 —CabDoréBunches—A photograph of Cabernet Doré showing a close up of three typical fruit cluster after version, Aug. 31, 2006.

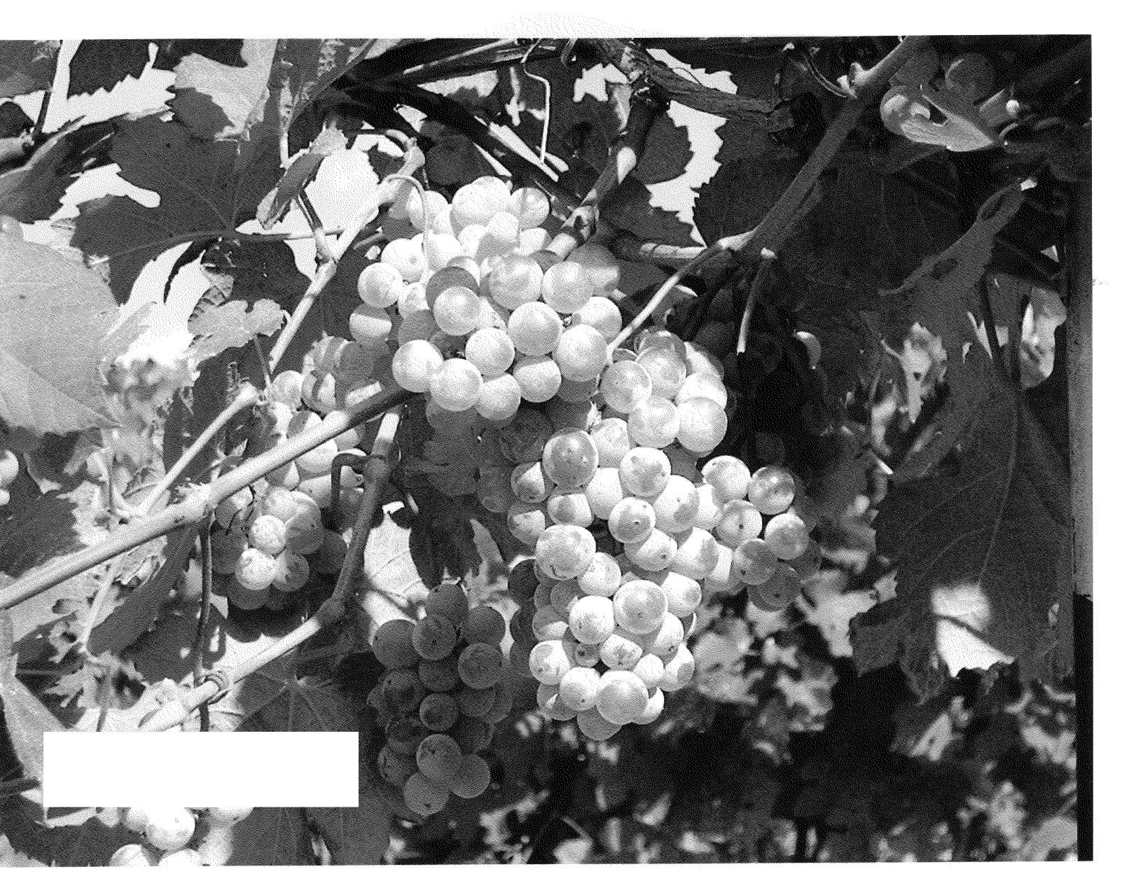

The colors in the photographs are as close as possible with the photographic and printing technology utilized. The color values cited in the detailed botanical description accurately describe the colors of the new grape.

### DETAILED BOTANICAL DESCRIPTION OF THE INVENTION

The following descriptions of Cabernet Doré apply to vines planted at the Mary Michelle Winery in Carrollton, Ill. in 2004. When dimensions, sizes, colors and other characteristics are given, it is to be understood that such characteristics and approximations set forth as accurately as possible. Variations of the usual magnitude incident to climatic factors, fertilization, pruning, pest control and other cultural practices are to be expected. Color codes used are those of The Royal Horticultural Society Colour Chart, copyrighted 2005.
Vine:
General:
   Size.—The test grapevines of Cabernet Doré are planted approximately 8 feet apart down the row and 10 feet apart between the rows. The vine canopy extends from 0.75 meters to 1.0 meters out in to the row. The vines were in their fourth leaf during the 2007 growing season.
   Vigor.—When first planted, much more vigorous than Norton. When mature equally vigorous as Cabernet Sauvignon but without the rampant and unruly growth habits of Norton.
   Productivity.—Productive, bearing 4 to 6 tons per acre depending on pruning and growing conditions.
Trunk:
   Surface texture.—Bark is loose, shaggy and peeling in long strips.
   Bark color.—Brownish. Varies from 200A to 202A.
   Trunk circumference.—Very straight, round, and uniform in diameter ranging from 2.2 cm to 2.4 cm at 40 cm height from ground to 1.9 cm to 2.2 cm at 600 cm from the ground.
Mature canes:
Size:
   Thickness.—Canes that arise from dormant spurs vary from 6 mm to 14 mm at 5 cm to 40 cm from bud. Canes are relatively uniform in width, like Norton and do not taper like Vidal.
   Surface.—Smooth often finely striated.
   Color.—Canes are reddish on top (45A) and light green underneath (145A).
   Internode length.—Varies widely depending on type of arm, position on vine and the nature of the bud from which each cane arose. From 3 cm to 4 cm on smaller shorter arms and laterals and from 10 cm to 15 on larger canes. Varies from 9 to 14 cm on the stronger upper suncanes and from 3 to 5 cm on lateral canes.
Tendrils: Medium in length ranging from 10 to 20 cm.
   Diameter.—Varies from 0.5 to 1.5 mm in thickness measured at the base of the tendril.
   Distribution.—Discontinuous.
   Form.—Predominately bifid, although trifid tendrils may be present.
   Color.—Brown (N167A).
Growing tips: Straight, and uniformly green (137C)
Leaves:
Mature leaves:
   General.—Like its parent Norton, the leaves of Cabernet Doré can have a wide variety of shapes and sizes on the same plant. Variations can be caused by the age of the vine, the location where grown, the weather and any number of other factors.
   Average blade length.—9 cm as measured from the apex of the center leaf lobe to the petiole junction.
   Size of blade.—Small to medium, usually lobed. Often resembles a small Cabernet Sauvignon leaf with overlapping superior lobes appearing to create holes in the leaf surface resembling eyes.
   Shape.—Cuneo-truncate. Galet coordinates: 146-3-57.
   Shape of teeth along leaf margins.—Convex.
   Length of teeth along leaf margins.—Small to medium and variable, 2 to 4 mm in length.
   General shape of petiole sinus.—Open, but spear shaped, narrowing at base.
   Shape of upper leaf sinus.—Lightbulb shaped when open. When closed has appearance of round hole in surface of leaf.
Leaf color:
   Upper leaf surface.—Rough (136A).
   Lower leaf surface.—Rather dull and lighter in intensity of color (138D).
   Upper leaf veins.—Yellowish-green (2C).
   Lower leaf veins.—Yellow-green (2C).
   Surface texture.—Rough.
   Surface appearance.—Dull.
   Lower surface texture.—Rough, dull.
Leaf petiole:
   Length of petiole.—6 cm to 8 cm.
   Petiole thickness.—2.0 to 3.0 mm measured at mid petiole.
   Petiole shape.—Round, glabrous, smooth.
   Petiole color.—Pinkish-red (RHS N57A).
   Length of petiole compared to mid vein.—on average about 70%.
Floral cluster description:
   Bloom timing.—Varies widely from year to year. Generally late, slightly after Norton. May 10^{th} in 2006.
   Cluster form.—conical to triangular.
   Clusters per shoot.—usually three.
   Floral cluster length.—average 80 cm.
   Floral cluster width.—average 30 cm.
   Cluster peduncles.—length 10 to 12 cm, thickness 3.0 to 4.0 cm.
   Inflorescence.—Hermaphroditic.
   Floral stamens.—Upright with normal size anthers.
   Pollen amount.—Abundant.
   Pollen color.—Pale yellow.
   Calyptras separation from the flower base.—Complete.
   Duration of bloom.—Average 10 to 12 days depending on ambient temperatures during the bloom period.
Fruit description—primary clusters:
   Date of maturity.—Sep. 15, 2006 in west central Illinois.
   Bunch size.—medium.
   Bunch length.—12 to 25 cm, not including the peduncle.
   Bunch width.—10 to 15 cm.
   Bunch form.—Triangular to round in shape. Usually well filled out with small to medium tight bunches.
   Bunch weight.—Average from 90 to 150 gm.
   Bunch density.—Tight like Norton.
   Peduncle length.—Medium from 3 to 5 cm.
   Peduncle thickness.—Ranges from 2.0 to 3.0 cm at the peduncle base.
   Berry form.—Round.
   Cross sectional view berry form.—Globose.
   Berry size.—small to medium 10 to 12 mm in diameter.
   Berry weight.—2 to 5 gm.
   Berry uniformity.—Excellent.
   Berry pedicle.—small to medium size.
   Length.—5 to 6 mm.
   Thickness.—1 to 2 mm.
   Attachment.—Very strong with no shatter at commercial maturity.
   Berry skin color.—Yellow to gold with waxy bloom. 100% colored.
   Berry skin thickness.—Medium.
   Berry skin surface texture.—Smooth and glabrous.
   Berry skin, tenacity to flesh.—Skin is tenacious to the flesh.
   Berry skin, tendency to crack.—Has never shown any cracking.
   Berry skin, reticulation.—Surface is smooth with no reticulations present.
   Berry color.—Light yellow brown (RHS 163B). With waxy bloom berries have a whitish look.
   Berry surfaces.—Uniformly covered with a waxy, whitish bloom.
   Berry flesh color.—Light translucent yellow (RHS 158D).
   Juiciness of flesh.—Similar to standard commercial wine varieties. Much juicier than Norton, not as juicy as Sauvignon Blanc.
   Berry firmness.—Relatively firm.
   Berry juice.—clear, light at crushing.
   Solids-sugar percentage (at maturity).—22.0 on Sep. 12, 2006.
   pH of berry juice.—3.18 on Sep. 12, 2006.
   Titratable acidity.—7.75 gm/liter.
   Seed.—Viable, 3 to 4 per berry, average size and shape for V. Vinifera.
   Flavor.—Good, tart, sweet, typical V. Vinifera white wine grape flavor.
   Aroma.—Typical crushed white wine grape aroma. No wild aromas.
Secondary bunches: Almost no secondary bunches have been observed in normal years with no spring frost.
Comparison between parental and commercial cultivars: The physical appearance of the vine of Cabernet Doré more closely resembles that of its parent Cabernet Sauvignon. Like Cabernet Sauvignon the leaves of Cabernet Doré are more modest in size and show fewer variations than do the leaves of its parent Norton. The growth habits are more orderly than Norton and it can produce normal crops of between 4 to 6 tons per acre without having to be grown on a double curtain trellis system. Cabernet Doré is far more resistant to the endemic vine diseases of the eastern US than Cabernet Sauvignon and it can be grown on its own roots. The berries and bunches are larger than Norton and the berries have fewer seeds making wine making easier.

What is claimed is:

1. The new and distinct variety of grape vine herein described and illustrated and identified by the characteristics enumerated above (application Ser. No. 11/978,502).
